(19) United States
(12) Patent Application Publication
(10) Pub. No.: US 2023/0357693 A1
(43) Pub. Date: Nov. 9, 2023

(54) MICROCOSMIC CULTURE DEVICE AND ITS APPLICATION IN QUANTITATIVE ANALYSIS OF SOIL CARBON DIFFUSION AND MICROBIAL UTILIZATION PROCESSES

(71) Applicant: BEIJING UNIVERSITY OF TECHNOLOGY, Beijing (CN)

(72) Inventors: Lu Yu, BEIJING (CN); Sichen Chen, BEIJING (CN)

(21) Appl. No.: 18/221,894

(22) Filed: Jul. 14, 2023

Related U.S. Application Data
(63) Continuation of application No. PCT/CN2022/088900, filed on Apr. 25, 2022.

(30) Foreign Application Priority Data
May 20, 2021 (CN) 202110553103.0

(51) Int. Cl. C12M 1/00; C12M 1/34; G01N 33/24

(52) U.S. Cl. CPC C12M 29/04; C12M 41/34; C12M 41/30; G01N 33/24

(57) ABSTRACT

The invention relates to the field of soil process analysis, in particular to a microcosmic culture device and its application in quantitative analysis of soil carbon diffusion and microbial utilization process. The microcosmic culture device comprises a closed container, an incubator and a dialysis tube in the closed container; The incubator comprises a soil layer; The dialysis tube is connected with the incubator, and part of the tube extends through the side wall of the incubator into the soil layer along the length direction. The dialysis tube is equipped with a carbon source, and the dialysis tube can make the carbon source spread to the soil layer, and always maintain the same water potential inside and outside the dialysis tube. The invention provides a quantitative analysis method for soil carbon diffusion and microbial utilization process based on the microcosmic culture device, through which the relationship between the efficiency of microbial utilization of exogenous carbon source and space can be explored, and the influence of the distance of carbon diffusion on the efficiency of microbial utilization of exogenous carbon can be further quantitatively analyzed.

### CROSS REFERENCE TO RELATED APPLICATION

This application is a continuation application of International application No.

PCT/CN2022/088900, filed on Apr. 25, 2022, titled “A Microcosmic Culture Device and its Application in Quantitative Analysis Of Soil Carbon Diffusion and Microbial Utilization Processes”, which claims the priority benefit of Chinese Patent Application No. 202110553103.0, filed on May 20, 2021. The contents of the above identified applications are hereby incorporated by reference in its entirety.

### TECHNICAL FIELD

The invention relates, in general, to the field of soil process analysis, and in particular to a microcosmic culture device and its application in quantitative analysis of soil carbon diffusion and microbial utilization process.

### BACKGROUND

As the main source and sink, soil organic carbon accounts for about 58% of the total soil carbon. In current studies, researchers often focus on carbon sequestration, in which carbon from plants is sequestered as soil organic carbon. In recent years, studies have shown that this process is not a simple polymerization of monomers into complex bodies, but a complex process involving physical and chemical interactions at the molecular level. In addition to carbon sequestration, carbon utilization has gradually become the focus of attention.

Some of the carbon in soil can be used directly by microorganisms, and some can be used by chemical reactions. However, a large part of the carbon source cannot be captured or used by microorganisms due to various factors. In fact, soil organic carbon utilization is a reaction process under the joint action of many factors, including abiotic factors, biological physiological factors, community dynamics and so on.

Based on the premise that the space distance between Carbon source and microorganism is an important factor affecting soil organic carbon utilization, scholars at home and abroad have either established a Microbial-Mineral Carbon Stabilization (MIMIC) calculation model or a submatter-microbial/microbial-substrate conceptual model in recent years. However, The quantitative research on microbial utilization of soil organic carbon lacks relevant methods and equipment.

### SUMMARY

In order to solve the problems existing in the prior art, the invention provides a microcosmic culture device and its application in quantitative analysis of soil carbon diffusion and microbial utilization process.

First, the invention provides a microcosmic culture device, including:

A closed container and an incubator and dialysis tube in the closed container;

The incubator comprises a soil layer;

The dialysis tube is connected with the incubator, and part of the tube body extends along the length through the side wall of the incubator into the soil layer.

Further, the dialysis tube is made of a selective dialysis membrane with a threshold size of 12-14kD, and passes through two side walls of the incubator. Preferably, the dialysis tube passes through the side walls of two opposite sides of the incubator; Preferably, the dialysis tube is parallel to the untraversed side wall and is the same distance from both untraversed side walls.

Further, the side wall of the incubator is a sterile plate.

Further, the soil in the soil layer is evenly laid.

Secondly, the invention provides an application of the microcosmic culture device in quantitative analysis of soil carbon diffusion or microbial utilization processes.

Further, the microcosmic culture device is used for microbial culture, and the soil carbon diffusion or microbial utilization process is quantitatively analyzed through the change of CO₂ concentration in the air and soil in the closed container.

Further, prior to quantitative analysis, the soil in the soil layer in the microcosmic culture facility underwent a pre-treatment process that included:

The ring knife method was used to measure the bulk density, remove the plant residues and small stones, air dry in a ventilated and cool place, and grind to a 2 mm sieve; Basic physicochemical properties were tested, including pH, bulk density, carbon, nitrogen and water potential.

Further, the applications include:

The microcosmic culture device was used for microbial culture, and the treatment group and the control group were set up. Glucose or ¹⁴C-glucose was loaded into the dialysis tube in the treatment group, and no carbon source was added into the dialysis tube in the control group.

The process of soil carbon diffusion or microbial utilization was quantitatively analyzed by the determination of microbial biomass carbon from multiple soil samples in the treatment group and the control group.

Further, the amount of microbial biomass carbon in the test is: the amount of microbial biomass carbon in the test is detected by substrate induced respiration method or chloroform extraction method.

Further, the substrate induced breathing method includes:

The autologous yeast extract of 12-14 g⋅ L⁻¹ was mixed at the ratio of 8-10 g fresh soil to 20 ml yeast solution and incubated in a closed sterilized bottle, during which the oscillations were reciprocated at a rate of 180-200 rpm. At 0, 30, 60, 120 and 180 min, the gas in the bottle was collected by syringe and CO₂ concentration was immediately determined by infrared gas analyzer (Li820, Licor Biosciences), and the amount of microbial biomass carbon was detected by linear regression analysis.

Further, the chloroform extraction method includes:

After 30 to 40 minutes of chloroform extraction, glass fiber filtration and compressed air bubble removal of excess chloroform, the samples to be tested were obtained. After freezing, total organic carbon was determined by Shimadzu TOC-V (TOC-V). The microbial biomass carbon was calculated by minus the chloroform treated group. In addition, three blanks were set in the experiment to correct the background value.

Further, the soil samples with different distances from the dialysis tube are obtained as follows: The soil samples with different distances from the dialysis tube are obtained according to the fixed spacing, which is 0.25-1 cm.

For example, with spacing of 0.5 cm or 1.0 cm, 0-0.5 cm, 0.5-1 cm and 1.0-2.0 cm, each spatial location should be randomly sampled at least 5 times. Evenly mixed samples should be regarded as samples representing the spatial location.

Furthermore, glucose polymer was added to the dialysis tubes in the treatment group and the control group to maintain the balance of water potential inside and outside the dialysis tubes.

Further, the glucose polymer is dextran.

The invention has the following beneficial effects:

Based on the space distance, an important abiotic factor, the biological material dialysis tube is selected as a physical barrier device between carbon source and microorganism to achieve the goal of selective penetration, and a microcosmic culture device which can be used for quantitative analysis of soil carbon diffusion and microbial utilization process is obtained.

The invention adds dextran to the dialysis tube as a microcirculation dredge agent, which can ensure that the water potential in the dialysis tube is consistent with the water potential of the soil solution, and avoid the mass flow effect affecting the diffusion movement of carbon; The invention also utilizes isotope marking means 14C for quantitative analysis, which has significant effect on quantitative study of carbon diffusion process and microbial response.

The establishment of quantitative methods and devices for carbon diffusion and microbial utilization law laid a foundation for studying carbon diffusion law and microbial response mechanism, and also provided a new idea for improving the bioutilization efficiency of soil carbon.

### BRIEF DESCRIPTION OF DRAWINGS

FIG. 1 provides a microcosmic culture device for embodiment 1 of the invention;

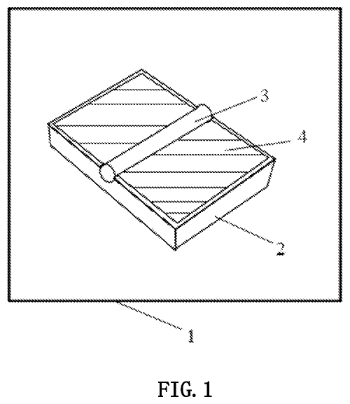

In FIG. 1: 1. Closed container; 2. Incubator; 3. Dialysis tube; 4. Soil layer.

### DETAILED DESCRIPTION OF THE PREFERRED EMBODIMENTS

The present disclosure will be further described below with the preferred embodiment, but the present invention is not limited to the following examples.

### Embodiment 1

The invention provides a microcosmic culture device, as shown in FIG. 1, which comprises a closed container 1, an incubator 2 and a dialysis tube 3 in the closed container 1;

The incubator 2 comprises a soil layer 4;

The dialysis tube 3 is connected with the incubator 2, and part of the tube body extends along the length through the side wall of the incubator 2 into the soil layer 4.

Airtight container 1 can be selected from a variety of airtight containers commonly used in this field, as long as the air tightness is maintained, such as a capped wide-mouth bottle.

Further, the dialysis tube 3 passes through two side walls of the incubator 2;

Preferably, the dialysis tube 3 passes through the lateral walls of two opposite sides of the incubator 2;

Further preferably, the dialysis tube 3 is parallel to the untraversed side wall and is the same distance from both untraversed side walls. In the case of the same distance, other factors except spatial distance are ensured to be relatively unchanged, making it easier to control other variables to ensure the accuracy of exploring the efficiency of microorganisms' utilization of exogenous carbon sources and the spatial relationship.

Further, the side wall of the incubator 2 is a sterile plate to prevent the influence of miscellaneous bacteria on the experimental results.

Further, the soil in the soil layer 3 is evenly laid.

In practical application, the microcosmic culture device can be used for quantitative analysis of soil carbon diffusion or microbial utilization process, specifically including:

The microcosmic culture device was used for microbial culture, and the treatment group and the control group were set up. Glucose or ¹⁴C-glucose was loaded into the dialysis tube in the treatment group, and no carbon source was added into the dialysis tube in the control group.

Soil samples with different distances from dialysis tubes were obtained and microbial biomass carbon content was detected.

The process of soil carbon diffusion or microbial utilization was quantitatively analyzed by the determination of microbial biomass carbon from multiple soil samples in the treatment group and the control group.

Among them, the soil samples with different distances from the dialysis tube can be obtained in various ways, such as 0.5 cm or 1.0 cm as spacing, 0-0.5 cm, 0.5-1 cm, 1.0-2.0 cm, random sampling at each spatial location at least 5 times, evenly mixed samples as representative of the spatial location of the sample.

### Embodiment 2

Based on the microcosmic culture device provided in Embodiment 1, this embodiment provides a quantitative analysis method for soil carbon diffusion and microbial utilization processes, specifically including the following flow:

The bulk density and field water capacity of some dryland soil samples were measured. Plant residues and small stones were removed from the remaining soil samples and ground to a 2 mm screen to obtain the soil for test. Soil pH, carbon and nitrogen, water content and the bulk density of the original soil were measured. Deionized water was added to make the soil moisture content 65% of the field water capacity. The water potential measurement system was used to measure the soil water potential. According to the soil water potential, the increment of Dextran in the dialysis tube (made of a selective dialysis membrane with a threshold size of 12-14kD) was calculated, and the increment of dextran was added, specifically referring to the water potential of the solution of ψ_{DEX}=−22.5[DEX]²−1.4[DEX] (ψ_{DEX,} Dextran 40; [DEX], Dextran 40 solution concentration). Three treatments were set up. The first was loaded into the dialysis tube with ordinary glucose, the second was loaded into the dialysis tube with ¹⁴C-glucose, and the third was loaded into the dialysis tube without carbon source (control group). The experiment was carried out according to the following steps:

1. CO₂ test

The dialysis tube was placed in the center of the incubator, soil was evenly laid on both sides, and the incubator was surrounded by sterile boards. The completed incubator was placed in a sterile wide-mouth bottle and sealed for culture. Culture for 8 days, during which the concentration of CO₂ or ¹⁴C-CO₂ in the bottle was monitored in real time.

2. Substrate induced respiration method to detect the amount of microbial biomass activated carbon

The dialysis tube was placed in the center of the incubator, soil was evenly laid on both sides, and the incubator was surrounded by sterile boards. The completed incubator was placed in a sterile wide-mouth bottle and sealed for culture.

The above culture devices were equipped with multiple devices, and the open-cover sampling of any device was randomly selected regularly. The sampling standard was divided into three sub-samples according to the distance from the dialysis tube: 0-0.5 cm soil sample, 0.5-1.0 cm soil sample and 1.0-2.0cm soil sample. The substrate-induced respiration method was used to determine the microbial biomass activated carbon of each soil sample, specifically as follows: The mixture was thoroughly mixed with 8 g fresh soil/20 ml yeast solution and cultured in a closed sterilized bottle, during which the oscillations were reciprocated at 180 rpm. At 0, 30, 60, 120 and 180 minutes, the gas in the bottle was collected by syringe and CO₂ concentration was immediately determined by infrared gas analyzer (Li820, Licor Biosciences), and then converted into microbial biomass activated carbon by linear regression analysis.

3. Chloroform extraction method to detect the amount of microbial biomass activated carbon

The dialysis tube was placed in the center of the incubator, soil was evenly laid on both sides, and the incubator was surrounded by sterile boards. The completed incubator was placed in a sterile wide-mouth bottle and sealed for culture.

The above culture devices were equipped with multiple devices, and the open-cover sampling of any device was randomly selected regularly. The sampling standard was divided into three sub-samples according to the distance from the dialysis tube: 0-0.5 cm soil sample, 0.5-1.0 cm soil sample and 1.0-2.0 cm soil sample. Chloroform extraction method was used to determine the microbial biomass carbon of each soil sample, specifically as follows: The comparative treatment with chloroform and without chloroform was set. The liquid to be tested was obtained through 30 minutes of chloroform extraction, glass fiber filtration, compressed air bubble removal and other steps. After freezing, the total organic carbon was determined by Shimadzu TOC-V. The group treated with chloroform minus the group treated without chloroform was then converted into microbial biomass carbon by relevant parameters.

4. Explanation of experimental results:

(1) CO₂ test

In the 8-day culture experiment, the CO₂ emission curve of the control group was y=0.1865×−0.0452 (R²=0.9816), and that of the carbon source group was y=0.2219×−0.0719 (R²=0.9811). The CO₂ emission rate of the carbon source group was significantly higher than that of the control group. From the second day, CO₂ emissions in the carbon 10 source group were significantly higher than those in the control group, exceeding 13.5%. On the 8th day, the amount of CO₂ in the carbon source group and the control group still did not reach the peak, indicating that there was enough carbon source for microbial utilization, and that the top space of the culture facility was sufficient for accurate measurement of CO₂ value.

(2) microbial biomass carbon

The separation and placement of carbon sources from soil did not affect the utilization of foreign carbon sources by soil microorganisms, which was shown in that the microbial biomass carbon in the carbon source group was significantly higher than that in the control group. In addition, soil microorganisms' use of carbon sources presents a distance gradient rule, which can be shown as follows: The increment of 0-0.5 cm soil microbial biomass carbon was 70-106 mg kg⁻¹, 0.5-1.0 cm soil microbial biomass carbon was 24-38 mg kg⁻¹, and 1.0-2.0 cm soil microbial biomass carbon was 1.0-4.0 mg kg⁻¹. It also indicates that the method and culture device of the invention are suitable for the study of carbon diffusion and utilization of microorganisms.

(3) ¹⁴C-Microbial biomass carbon

The ¹⁴C-microbial biomass carbon showed a gradient pattern, and the ¹⁴C-microbial biomass carbon closer to the carbon source (0-0.5 cm) was significantly higher than the ¹⁴C-microbial biomass carbon farther away (0.5-1.0 cm and 1.0-2.0 cm). Compared with the control group, the microbial biomass carbon was: The increment of ¹⁴C-microbial biomass carbon in 0-0.5 cm soil was 0.0110-0.0160nmol, and that in 0.5-1.0 cm soil was 0.0010-0.0021nmol. The increment of ¹⁴C-microbial biomass carbon in 1.0-2.0 cm soil was 0.0005-0.0010nmol. This confirms that microorganisms can utilize exogenous carbon sources as described in Result 2, and utilization efficiency is correlated with spatial location. Carbon diffusion distance affects microbial utilization efficiency of carbon.

Although the invention has been described in detail by the general description and the specific implementation scheme above, it is obvious to the technical personnel in the field that some modifications or improvements can be made on the basis of the invention. Therefore, the modifications or improvements made on the basis of not deviating from the spirit of the invention are within the scope of protection required by the invention.

What is claimed is:

1. A microcosmic culture device for quantitative analysis of soil carbon diffusion or microbial utilization processes, comprising: a closed container, an incubator and a dialysis tube in the closed container; wherein
the incubator comprises a soil layer in which soil is evenly laid;
the dialysis tube is connected with the incubator, and part of a tube body of the dialysis tube extends into the soil layer along a length direction through side walls of two opposite sides of the incubator, and the dialysis tube is parallel with two unpenetrated side walls of the incubator and has the same distance with the two unpenetrated side walls; the dialysis tube is made of a selective dialysis membrane with a threshold size of 12-14kD, the dialysis tube is equipped with a carbon source which can be diffused to the soil layer, dextran is added to the dialysis tube as a microcirculation dredge agent to ensure that water potential in the dialysis tube is consistent with that in soil solution.

2. The microcosmic culture device according to claim 1, wherein the side walls of the incubator is a sterile plate.

3. Use of the microcosmic culture device of claim 1 in quantitative analysis of soil carbon diffusion or microbial utilization processes.

4. Use of the microcosmic culture device according to claim 3, comprising cultivating microorganisms using the microcosmic culture device and quantitatively analyzing soil carbon diffusion or microbial utilization processes through changes in CO₂ concentrations in air and soil in the closed container.

5. Use of the microcosmic culture device according to claim 4, comprising setting up a treatment group and a control group, the dialysis tube in the treatment group is loaded with glucose or ¹⁴C-glucose, and the dialysis tube in the control group is not loaded with carbon source; soil samples with different distances from dialysis tubes are obtained and microbial biomass carbon content is detected; the process of soil carbon diffusion or microbial utilization is quantitatively analyzed by determination of microbial biomass carbon from multiple soil samples in the treatment group and the control group.

6. Use of the microcosmic culture device according to claim 5, wherein an amount of microbial biomass carbon in the test is: the amount of microbial biomass carbon in the test is detected by substrate induced respiration or chloroform extraction.

7. Use of the microcosmic culture device according to claim 5, the soil samples obtained at different distances from the dialysis tubes are: the soil samples obtained at different distances from the dialysis tube are obtained at fixed distances of 0.25 to 1 cm.

8. Use of the microcosmic culture device of claim 2 in quantitative analysis of soil carbon diffusion or microbial utilization processes.

9. Use of the microcosmic culture device according to claim 8, comprising cultivating microorganisms using the microcosmic culture device and quantitatively analyzing soil carbon diffusion or microbial utilization processes through changes in CO₂ concentrations in air and soil in the closed container.

10. Use of the microcosmic culture device according to claim 6, the soil samples obtained at different distances from the dialysis tubes are: the soil samples obtained at different distances from the dialysis tube are obtained at fixed distances of 0.25 to 1 cm.
